(19) United States
(12) Patent Application Publication
(10) Pub. No.: US 2004/0049803 A1
(43) Pub. Date: Mar. 11, 2004

(54) Senescence-associated plant promotors

(76) Inventors: Jill A. Kellogg, Bend, OR (US); Debra K. Schuster, Portland, OR (US); Stephanie K. Clendennen, Kingsport, TN (US)

Correspondence Address: JAN P. BRUNELLE, EXELIXIS, INC., 170 HARBOR WAY, P.O. BOX 511, SOUTH SAN FRANCISCO, CA, 94083-0511, US

(21) Appl. No.: 10/450,358

(22) PCT Filed: Dec. 10, 2001
(86) PCT No.: PCT/US01/47387

(51) Int. Cl. A01H 1/00; C12N 9/22; C12Q 1/68; C07H 21/04; C12N 15/82; C12N 5/04

(52) U.S. Cl. 800/278; 435/69.1; 435/6; 435/199; 435/320.1; 435/419; 536/23.2; 800/287

(57) ABSTRACT

The present invention is directed to MTI and GS promoters capable of promoting the senescence-associated expression of nucleic acid coding sequences operably linked thereto, together with heterologous nucleic acid constructs, vectors, kits, transformation methods, transgenic plant cells and transgenic plants comprising such promoters.

### FIELD OF THE INVENTION

The present invention relates to novel senescence-associated promoters, MT1 and GS and to heterologous nucleic acid constructs, vectors, kits, and transformation methods employing such promoters. The invention further relates to transgenic plant cells and plants transformed with heterologous nucleic acid constructs comprising such senescence-associated promoters.

### REFERENCES

Adams, D. O., and Yang, S. F., Plant Physiology 70:117-123, 1977.

An, G, et al., EMBO J. 4:277-284, 1985.

Ausubel, F M, et al., in CURRENT PROTOCOLS IN MOLECULAR BIOLOGY, John Wiley and Sons, Inc., Media, Pa., 1992.

Altschul, S. F. et al., J. Mol. Biol. 215:403-410, 1990.

Altschul, et al., Nucl. Acids Res. 25(17), 3389-3402, 1997.

Becker, D., et al., Plant Mol. Biol. 20:1195-1197, 1992.

Bellini, C., et al., Bio/Technology 7(5):503-508, 1989.

Bhalla P L and Smith N, Molecular Breeding 4: 531-41, 1998.

Bleecker A B, Curr Opin Plant Biol 1(1), 73-8, 1998.

Bommineni V R et al., IN: PLANT GENETIC ENGINEERING: TOWARDS THE THIRD MILLENIUM (Ed.) A D Arencibia. Elsevier Science B.V. pages 206-214, 2000

Buchanan-Wollaston, V., Plant Physiol. 105: 839-46, 1994.

Buchanan-Wollaston, V and C. Ainsworth, Plant Mol. Biol. 33: 821-834, 1997.

Butt A et al., Plant J, 16(2):209-21, 1998.

Cabrera-Ponce J L et al., Plant Cell Reports 16: 255-260, 1997.

Catlin D et al., Plant Cell Reports 7: 100-103, 1988.

Clark D G, et al., In Kanellis et al., eds. BIOLOGY AND BIOTECHNOLOGY OFTHE PLANT HORMONE ETHYLENE II. Kluwer. pp. 357-364, 1999.

Clendennen, S K, et al., In BIOLOGY AND BIOTECHNOLOGY OF THE PLANT HORMONE ETHYLENE II. Eds: A K Kanellis, et al. Kluwer Academic Publishers. pp 371-380, 1999.

Comai, L., and Coning, A. J., U.S. Pat. No. 5,187,267, issued Feb. 16, 1993.

Cough, S J and Bent, A F, the Plant Journal 16(6): 735-743, 1998.

Deikman, J., et al., Plant Physiol. 100, 2013-2017, 1992.

Deikman, J., et al., Plant Mol. Biol. 37, 1001-1011, 1998.

Delbreil B et al., Plant Cell Reports 12:129-132

Fan L et al., Plant Cell, 9(12):2183-96, 1997.

Ferro, A, et al., U.S. Pat. No. 5,416,250, issued May 16, 1995.

Fils-Lycaon et al., Plant Physiol 111:269-273, 1996.

Franks T et al., Molecular Breeding 4:321-33, 1998.

Frary A, and Earle E D, Plant Cell Reports 16: 235-240, 1996.

Gan S et al., Science, 270(5244):1986-8, 1995.

Good et al., Plant Mol. Biol. 26:781-790, 1994.

Hardegger M et al., Molecular Breeding 4: 119-127, 1998.

Hanfrey, C et al., Plant Mol. Biol. 30:5 97-609, 1996.

Hong Y et al. Proc Natl Acad Sci USA, 97(15):8717-22, 2000.

Hooykaas, P J, and Schilperoot, R A; in TRENDS IN BIOCHEMICAL SCIENCES, International Union of Biochemistry and Elsevier Science Publishers, v.10(8):307-309, 1985.

Hosoki T et al., J. Japan Soc. Hort. Sci. 60: 71-75, 1991.

Hong Y et al, 2000, Proc Natl Acad Sci USA 97:8717-22.

Houck, C M and Pear, J R, U.S. Pat. No. 4,943,674, issued Jul. 24, 1990.

Hughes, J A, et al., J. Bact. 169:3625-3632, 1987a.

Hughes, J A, et al., Nucleic Acids Res 15:717-729, 1987b.

Jefferson et al., EMBO J 1987, 6(13):3901-7.

Klein, T. M., et al., PNAS USA 85(22):8502-8505, 1988.

Kramer, M. G. et al. (1997), in A. K. Kanellis, et al. (eds.), Biology and Biotechnology of the Plant Hormone Ethylene, Kluwer Academic Publishers, Boston, pp. 307-319

Maloney, M. M. et al., Plant Cell Reports 8:238-242, 1989.

Mathews H et al, Plant Cell Rep., 14:471476, 1995a.

Mathews H et al, In Vitro 31:36-43, 1995a.

McCormick et al., Plant Cell Reports 5:81-84, 1986.

Miguel CM et al., Plant Cell Reports 18: 387-93, 1999.

Miki, B. L. A., et al., PLANT DNA INFECTIOUS AGENTS (Hohn, T., et al., Eds.) Springer-Verlag, Vienna, Austria, pp. 249-265, 1987.

Mogilner N et al., Mol Plant Microbe Interact 6(5):673-5, 1993.

Nakamura Y et al., Plant Cell Reports 17:435440.

Ni, M et al., Plant J. 7:661-676, 1995.

Norelli et al., HortScience, 31:1026-1027, 1996.

Ochs G et al., Plant Mol Biol. 39(3):395-405, 1999.

Ori Net al., Plant Cell, 11(6):1073-80, 1999.

Pogson, B J et al., Plant Plysiol. 108: 651-657, 1995.

Puddephat I J et al., Molecular Breeding 2: 185-210, 1996.

Ranier et al., Bio/Technology 8:33-38, 1990

Robinson, H L and Torres, Calif., Sem. Immunol. 9:271-282, 1997.

Sagi et al., Biotechnology (N Y). 1995, 13(“5):481-5.

Sambrook J, et al., in MOLECULAR CLONING: A LABORATORY MANUAL, Cold Spring Harbor Laboratory Press, Cold Spring Harbor, N.Y., Vol. 2, 1989.

Sarmento G G et al., Plant Cell Tissue and Organ Culture 31: 185-193, 1992.

Schwarz-Sommer, et al., EMBO J 1992, 11(1):251-63

Scorza R et al., Plant Cell Reports 14: 589-92, 1995.

Stewart, C N Jr., and Via LE, BioTechniques, 14(5):748-749, 1993.

Trulson A J et al., Theor Appl Genet 73: 11-15, 1986

Van Eck J M, et al., Plant Cell Reports 14: 299-304, 1995

Watanabe A et al., Plant Mol Biol., 26(6):1807-17, 1994.

Wilkinson et al., Nature Biotechnol., 15: 444-7, 1997.

Xu, R, et al., Plant Mol. Biol. 31:1117-1127, 1996.

Yamada K et al, 1999, Plant Cell Physiol 40:198-204.

Zhang H X and Zeevaari J A D, Plant Cell Reports 18-64045, 1999.

Zhu, Q, et al., Plant Cell 7:1681-1689, 1995.

Zuo J. Chua N H, Curr Opin Biotechnol, 11(2):146-51, 2000.

### BACKGROUND OF THE INVENTION

When plants and plant part die, it may be due to environmental stress, aging or senescence. Various kinds of physiological senescence and death occur and may affect particular cells, tissues, organs, or the whole plant.

Senescence is a complex process that is controlled by multiple developmental and environmental signals. Molecular studies indicate that changes in gene expression are associated with the progression of the senescence process. In some cases, environmental factors, such as photoperiod and temperature play a role in the onset of senescence, however, typically plant senescence is viewed as an organized, genetically controlled process which facilitates the salvage of nutrients (Bleecker AB, 1998). In some plant varieties, the development of fruits and seeds is associated with senescence and frequently with the death of the rest of the plant. During senescence, respiration may increase for a period and the synthesis of proteins and nucleic acids ceases.

Ethylene biosynthesis takes place in the early stages of senescence. Ethylene biosynthesis has been demonstrated to stimulate the expression of certain genes, which encode hydrolytic enzymes. Such hydrolytic enzymes break down complex molecules to subunits that are transportable for reuse, e.g. amino acids, nucleotides, sugars, fatty acids, and other small organic molecules. The onset of senescence is characterized by degeneration of plant cells with an associated accumulation of metabolic breakdown products. Such breakdown involves the synthesis of senescence-associated enzymes including proteases, nucleases, lipases, amylases, and others, e.g., pectinases and cellulases, which are effective to complete the hydrolytic process.

Promoter activity and associated gene expression which occurs or primarily occurs, during later stages of fruit development and/or early stages of fruit ripening, is said to be ethylene regulated. In some tissues, exposure to only a small amount of ethylene may cause an avalanche of ethylene production in adjacent plants or plant tissues such as fresh produce. This autocatalytic effect can be very pronounced and lead to loss of fruit quality during transportation and storage. Ethylene biosynthesis in florets has been implicated in postharvest deterioration of Brassica oleraea (broccoli), especially floret yellowing caused by chlorophyll degradation in the sepals (Pogson et al., 1995).

The vegetable, fruit and cut flower industries are interested in interfering with the deleterious effects of ethylene on vegetables, fruit and cut flowers during transportation and storage in order to extend the postharvest shelf life of agricultural products.

Accordingly, there is a need for regulation of the senescence process in order to prolong the postharvest life of vegetables, fruit and cut flowers.

### SUMMARY OF THE INVENTION

The invention provides isolated senescence-associated promoters, characterized by the ability to promote expression of a gene to which the senescence-associated promoter sequence is operably linked. In a preferred embodiment, the senescence-associated promoter comprises a metallothionein (MT1) promoter sequence as presented in SEQ ID NO:1. The senescence-associated promoter may also comprise a nucleotide sequence having at least 95% sequence identity to the sequence presented as SEQ ID NO:1 or fragment thereof, which hybridizes to the sequence presented as SEQ ID NO:1 or a fragment thereof under high stringency conditions.

The invention further provides a plant expression vector comprising a senescence-associated promoter operably linked to a nucleic acid coding sequence.

In a related aspect, the invention provides plant cells and mature plants comprising the plant expression vector, as well as methods for modifying senescence-associated gene expression in a plant using such a vector.

In one preferred approach, the plant expression vector includes a nucleic acid coding sequence for a gene product associated with the delay of senescence, or an ethylene-associated gene product, where expression of the nucleic acid coding sequence interferes with the senescence of vegetables, fruits or flowers produced by the transgenic plant. S-adenosyl methionine hydrolase (SAMase) is provided as an exemplary nucleic acid coding sequence for use in practicing the invention.

### BRIEF DESCRIPTION OF THE FIGURES

FIG. 1 is a schematic representation of the exemplary pAG 4228 binary plasmid, which has the pPZP200 backbone, the sam-k coding sequence under the control of the senescence-associated MT1 promoter, and a nos termination element located downstream of the sam-k coding sequence adjacent the right border of the plasmid; and the nptII selectable marker coding sequence under the control of a raspberry RE4 promoter with an Agrobacterium gene 7 termination element located downstream of the nptII gene, adjacent the left border of the plasmid.

### DETAILED DESCRIPTION OF THE INVENTION

I. Definitions

Unless otherwise indicated, all terms used herein have the same meaning as they would to one skilled in the art of the present invention. Practitioners are particularly directed to Sambrook et al. (1989) Molecular Cloning: A Laboratory Manual (Second Edition), Cold Spring Harbor Press, Plainview, N.Y. and Ausubel a et A (1993) Current Protocols in Molecular Biology, John Wiley & Sons, New York, N.Y., for definitions and terms of the art. It is to be understood that this invention is not limited to the particular methodology, protocols, and reagents described, as these may vary.

As used herein, the term “polynucleotide” refers to a polymeric molecule having a backbone that supports bases capable of hydrogen bonding to typical polynucleotides, where the polymer backbone presents the bases in a manner effective to permit such hydrogen bonding in a sequence specific fashion between the polymeric molecule and a typical polynucleotide (e.g., single-stranded DNA). Polymeric molecules include double and single stranded ribonucleic acids (RNA) and deoxyribonucleic acids (DNA), and may include polymers having backbone modifications.

A nucleic acid may be double stranded, single stranded, or contain portions of both double stranded and single stranded sequence. The depiction of a single strand also defines the sequene of the other stranded and thus also includes the complement of the sequence which is depicted.

The term “polypeptide” as used herein refers to a compound made up of a single chain of amino acid residues linked by peptide bonds. The term “protein” as used herein may be synonymous with the term “polypeptide” or may refer, in addition, to a complex of two or more polypeptides. In the context of the present invention, a “protein complex” refers to multiple copies of the same protein or protein fragment that bind to a single ribonucleotide fragment. Generally, but not always., polypeptides and proteins are formed predominantly of naturally occurring amino acids.

Amino acid residues are referred to herein by their standard single letter notations: A, alanine; C, cysteine; D, aspartic acid; E, glutamic acid; F, phenylalanine; G, glycine; H, histidine; U, isoleucine; K, lysine; L, leucine; M, methionine; N, asparagine; P, proline; Q, glutamine; R, arginine; S, serine; T, threonine; V, valine; W, tryptophan; Y, tyrosine.

As used herein, the term “sequence identity” means nucleic acid or amino acid sequence identity between two or more aligned sequences, aligned using a sequence alignment program.

The term “% homology” is used interchangeably herein with the term “% identity” and refers to the level of nucleic acid or amino acid sequence identity between two or more aligned sequences, when aligned using a sequence alignment program. For example, as used herein, 80% homology means the same thing as 80% sequence identity determined by a defined algorithm, and accordingly a homologue of a given sequence has 80% or more sequence identity over a length of the given sequence. Exemplary levels of sequence identity include, but are not limited to, 80, 85, 90 or 95, 98% or more sequence identity to a given sequence.

A preferred alignment of two or more selected sequences in order to determine the “% identity” between the sequences is performed using for example, using the CLUSTAL W program in MacVector version 6.5, operated with default parameters, including an open gap penalty of 10.0, an extended gap penalty of 0.1, and a BLOSUM-30 similarity matrix.

Exemplary computer programs which can be used to determine identity between two sequences include, but are not limited to, the suite of BLAST programs, e.g., BLASTN, BLASTX, and TBLASTX, BLASTP and TBLASTNT, publicly available on the Internet at http//www.ncbi.nlm.nih.gov/BLAST/”. See, also, Altschul, S. F. et al., 1990 and Altschul, S. F. et al., 1997.

Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences which have been translated in all reading frames against amino acid sequences in the GenBank. Protein Sequences and other public databases. Both BLASTN and BLASTX are run using default parameters of an open gap penalty of 11.0, and an extended gap penalty of 1.0, and utilize the BLOSUM-62 matrix. [See, Altschul, et al., 1997.]

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° below the Tm; “intermediate stringency” at about 10-20° below the Tm of the probe; and “low stringency” at about 20-25° below the Tm. Functionally, maximum stringency conditions may be used to identify sequences having strict identity or near-strict identity with the hybridization probe, e.g., high stringency conditions are used to identify sequences having about 80% or more sequence identity with a reference nucleic acid sequence.

Moderate and high stringency hybridization conditions are well known in the art. (See, for example, Sambrook, et al., 1989, Chapters 9 and 11, and Ausubel, F. M., et al., 1993, expressly incorporated by reference herein). An example of high stringency conditions includes hybridization at about 42° C. in 50% formamide, 5×SSC, 5× Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C.

As used herein, the term “recombinant nucleic acid” refers to a nucleic acid, originally formed in vitro, generally by the manipulation of a nucleic acid sequence using endonucleases, where the recombinant nucleic acid is in a form not typically found in nature.

As used herein, the terms “chimeric” and “heterologous” relative to a “gene”, or “gene construct”, “nucleic acid sequence” or “nucleic acid construct” are used interchangeably and refer to recombinant nucleic acid sequences which include a DNA coding sequence and control sequences required for expression of the coding sequence in a plant cell.

As used herein, the term “transgene” refers to a nucleic acid sequence, usually encoding a polypeptide which is typically heterologous to the control sequences to which it is operably linked. In general, a transgene is introduced into a host genome using recombinant DNA techniques.

As used herein, the term “recombinant” as used with reference to a cell, means that the cell has been modified by the introduction of a heterologous nucleic acid sequence or that the cell is derived from a cell so modified. Thus, for example, recombinant cells express genes that are not found in an identical form within the native (non-recombinant) form of the cell. In some cases, a recombinant cell will exhibit abnormal expression, e.g., the gene is under expressed or not expressed at all as a result of deliberate human intervention.

As used herein, the term “plasmid” refers to a circular double-stranded (ds) DNA construct used as a cloning vector, and which forms an extrachromosomal self-replicating genetic element in many bacteria and in some eukaryotes.

As used herein, the term “vector” refers to a nucleic acid construct designed for transfer between different host cells. An “expression vector” refers to a vector that has the ability to incorporate and express a heterologous DNA fragment in a foreign cell. Many prokaryotic and eukaryotic expression vectors are commercially available. Selection of appropriate expression vectors is within the knowledge of those having skill in the art.

As used herein, the term “operably linked” relative to a heterologous nucleic acid construct, or vector means the nucleotide components of the construct or vector are in a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, “operably linked” means that the nucleic acid sequences being linked are contiguous, and in the case of a secretory leader, contiguous and in reading phase. However, in some cases, operably linked nucleic acid sequences are not contiguous, e.g., enhancers may not be contiguous.

As used herein, the term “gene” means the segment of DNA involved in producing a polypeptide chain, which may or may not include regions preceding and following the coding region, e.g. 5′ untranslated (5′ UTR) or “leader” sequences and 3′ UTR or “trailer” sequences, as well as intervening sequences (introns) between individual coding segments (exons). The term “gene”, may be used interchangeably herein with the term “nucleic acid coding sequence”, and the term “structural gene”.

A nucleic acid sequence is said to be “heterologous” with respect to a control sequence (i.e. promoter or enhancer) when it does not function in nature to regulate the same gene the expression of which it is currently regulating. Generally, heterologous nucleic acid constructs are introduced into a cell or part of the genome and have been added to the cell, by transfection, microinjection, electroporation, or the like. The sequences may contain a control sequence/DNA coding sequence combination that is the same as, or different from a control sequence/DNA coding sequence combination found in a native plant cell.

As used herein, the terms “promoter”, “promoter segment” and “promoter fragment” all refer to a sequence of DNA that functions to direct transcription of a downstream gene in the host cell in which the gene is being expressed. The promoter together with other transcriptional and translational regulatory nucleic acid sequences (also termed “control sequences”) are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

As used herein, the term “regulatable promoter” refers to any promoter whose activity is affected by specific environmental or developmental conditions (e.g., a tomato E4 or E8 promoter). A “regulatable promoter” may be inducible by a specific environmental signal, e.g., the onset of senescence.

As used herein, the term “constitutive promoter” refers to any promoter that directs RNA production in a transformed plant cell and/or many or all tissues of a transformed plant at most times.

As used herein, the term “border sequence” refers to the nucleic acid sequence which corresponds to the left and right edges (“borders”) of a T-DNA sequence.

As used herein, the terms “transformed”, “stably transformed” or “transgenic” with reference to a plant cell means the plant cell has a non-native (heterologous) nucleic acid sequence integrated into its genome which is maintained through two or more generations.

As used herein, the term “expression” refers to the process by which a polypeptide is produced based on the nucleic acid sequence of a gene. The process includes both transcription and translation.

The term “introduced” in the context of inserting a nucleic acid sequence into a cell, means “transfection”, or “transformation” or “transduction” and includes reference to the incorporation of a nucleic acid sequence into a eukaryotic or prokaryotic cell. The nucleic acid sequence may be incorporated into the genome of the cell (for example, chromosome, plasmid, plastid, or mitochondrial DNA), converted into an autonomous replicon, or transiently expressed (for example, transfected mRNA).

As used herein, a “plant cell” refers to any cell derived from a plant, including undifferentiated tissue (e.g., callus) as well as plant seeds, pollen, progagules and embryos.

As used herein, the term “mature plant” refers to a fully differentiated plant.

As used herein, the terms “native” and “wild-type” relative to a plant trait, phenotype, plant cell or plant refers to the form in which that trait, phenotype, plant cell or plant is found in the same variety of plant in nature. Hence, a “native” or “wild-type” plant cell or plant is non-transgenic.

As used herein, the term “transgenic plant” refers to a plant that has incorporated a heterologous or exogenous nucleic acid sequence, i.e., a nucleic acid sequence which is not present in the native (non-transgenic or “untransformed”) plant or plant cell. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic plant. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, the term “modulate” refers to a change in biological activity. Modulation may relate to an increase or a decrease in biological activity, binding characteristics, or any other biological, functional, or immunological property of the molecule.

As used herein, the term “undergoing senescence” refers to a biological process that takes place in vegetative tissues, flowers and fruit, where senescense is associated with yellowing, usually the result of chlorophyll breakdown, loss of turgor and membrane integrity leading to dessication, wilting and browning, loss of tissue firmness and integrity, abcission of leaves and petals, and general oxidative damage, as described, for example in Hong et al., 2000.

Accordingly, a senescence phenotype, as defined herein refers to a plant which exhibits the characteristics of senescence in one or more of vegetative tissues, flowers and fruit, e.g., yellowing, loss of turgor and membrane integrity, wilting and browning, loss of tissue firmness and integrity, abcission of leaves and petals, and general oxidative damage.

As used herein, the term “modulation of the senescence process” means a change in progression to a senescence phenotype, e.g., inhibition of the senescence process evidenced by a slowing, partial or complete inhibition of the development to a senescence phenotype.

As used herein, the term “gene product associated with the delay of senescence” refers a gene product that may interfere with ethylene biosynthesis and perception, and may also interefere with the action of enzymes that are involved in the loss of cellular integrity, oxidation, cell wall degradation or abcission. The gene product may delay, diminish, and/or eliminate the senescence phenotype in plants or plant parts, including leaves, vegetables, fruits, and/or flowers. Examples include an ethylene-insensitive ethylene receptor, S-adenosylmethionine hydrolase (SAMase), SAM decarboxylase, the homeobox gene knotted-1 (Ori et al., 1999), and genes that induce cytokinin production (Gan & Amasino, 1995). The genetic manipulation of senescence may also be effected by the suppression of senescence-associated genes such as lipases and phospholipases (Fan et al., 1997; Hong et al., 2000).

As used herein, the term “ethylene regulated”, refers to a plant process induced by changes in ethylene concentration in the plant. For example, promoter activity and associated gene expression which occurs or primarily occurs, during later stages of fruit development and/or early stages of fruit ripening, is said to be ethylene regulated.

As used herein, the term an “ethylene-associated gene product” refers to a gene product which can affect the production or metabolism of ethylene or the response of a plant to ethylene.

II. Senescence in Plants and Ethylene Regulation

The present invention is directed to plant regulatory nucleic acid sequences involved in senescence and the use of such regulatory nucleic acid sequences in modulating the senescence process.

Senescence has been defined as part of the plant aging process leading to organ and/or plant death. As the developmental cycle of plants proceeds and/or in response to environmental stress, the senescence process takes place and nutrients are mobilized to developing parts of the plant, such as growing leaves, developing flowers, seeds and to other storage organs. Senescence is an active metabolic process involving the production of a number of enzymnes typically accompanied by increased respiration rate.

Ethylene biosynthesis takes place in the early stages of senescence. Ethylene biosynthesis has been demonstrated to stimulate the expression of certain genes, which encode hydrolytic enzymes. Such hydrolytic enzymes break down complex molecules to subunits that are transportable for reuse, e.g. amino acids, nucleotides, sugars, fatty acids, and other small organic molecules. Senescence-associated enzymes include proteases, nucleases, lipases, amylases, and others, e.g., pectinases and cellulases which are effective to complete the hydrolytic process.

As the process of senescence takes place, the appearance of the plant changes. Plant tissues lose their characteristic green color, becoming more yellow and red in color. As the senescence progresses, plant tissue may become softer with a loss of volume and shriveling of plant tissue may become evident.

A. Modified Senescence Phenotype

The availability of the plant senescence-associated promoters of the invention makes possible the generation of genetic constructs that interfere with (delay, diminish, and/or eliminate) the normal senescence phenotype (i.e., relative to the corresponding non-transgenic plant) of transgenic plants into which they are introduced. Senescence of plants or plant parts, including leaves, vegetables, flowers, and/or fruits may be affected. Genetic constructs comprising a transgene operably linked to a senescence-associated promoter of the invention can be introduced into plant cells and used to develop transgenic plants, such that expression of the transgene takes place upon the plant entering senescence. Such senescence-associated expression permits the expression of genes which might be disruptive of plant morphology or productivity if expressed at another stage of plant development and/or for which senescence-associated expression is advantageous.

One aspect of the present invention is a nucleic acid construct comprising a senescence-associated promoter operably linked to a heterologous nucleic acid coding sequence that is not associated with the promoter in a native plant. Useful senescence-associated promoters, identified herein as the MT1 and GS promoters, have been characterized. The senescence-associated promoters of the invention enable the creation of transgenic plants with a modified senescence phenotype, e.g., delayed senescence.

For example, the coding sequence for a gene product associated with the delay of senescence and/or a slowing of the ripening process (e.g., in vegetables, fruits, or flowers) may be placed under the control of a senescence-associated promoter, thereby prolonging the useful postharvest life of vegetables, fruits and flowers.

Although the mechanism is not part of the invention, it will be appreciated that at the onset of senescence, a senescence-associated promoter becomes active resulting in expression of a gene operably linked thereto. Such senescence-associated gene expression may be used to alter the progression of senescence in a transgenic plant.

Ethylene is a plant hormone influencing many aspects of plant growth and development, and is known to play a major role in the ripening process in vegetables and fruits. Accordingly, a transgene involved in the production of ethylene or regulation of the ethylene response in plants may be operably linked to a senescence-associated promoter of the invention and thereby modulate the effects of ethylene on the ripening process in vegetables, fruits and flowers. Various gene products are involved in ethylene production and regulation in plants, as further described below.

A transgenic plant with a modified senescence phenotype typically comprises a heterologous nucleic acid construct which has a transgene expressed under the control of a senescence-associated promoter of the invention. Such a transgenic plant exhibits an alteration in the senescence process, which is generally characterized by a progressive loss of green leaf color, increased leaf and sepal yellowing, and/or loss of tissue volume and/or shriveling, relative to a corresponding (i.e., non-transgenic) wild-type plant. Exemplary modifications include a change in the time course and/or a decrease in, or lack of, the characteristic senescence phenotype in a transgenic plant relative to a corresponding (i.e., non-transgenic) wild-type plant.

III. Senescence-Associated Promoter Compositions of the Invention

A. Senescence-Associated Promoters

Transcriptional regulatory sequences or promoters that regulate gene expression in plants are essential elements of plant genetic engineering. Most promoters are from about 500-1500 bases in length and numerous examples of promoters useful for the expression of heterologous genes in plants are now available (Zhu, et al., 1995; Ni, et al., 1995).

Promoters that direct tissue-associated gene expression, e.g., direct RNA synthesis at higher levels in particular types of cells and/or tissues, e.g., fruit-associated promoters have been described. Examples of fruit-associated promoters include a cherry 29 (CH29) promoter (Fils-Lycaon et al., 1996); an mf6O promoter (Yamada et al., 1999); a Thi::actin or aThi 1.3::intron promoter (described in co-owned 09/560,419, expressly incorporated by reference herein); a dru 1.3 promoter (described in co-owned U.S. Pat. No. 5,783,394, expressly incorporated by reference herein); a MADS2 promoter, a fuji Thi 1.3 and a Thi 1.0 fruit-associated promoter (described in co-owned U.S. S No. 60/132,124, expressly incorporated by reference herein); melon fruit-associated promoters including a cmACO1/TE4 promoter, a MEL7 promoter, a MEL2 promoter, a 6E promoter and a 2F promoter (described in U.S. S No. 60/190,414, expressly incorporated by reference herein).

Other promoters direct gene expression in response to developmental changes in the plant or plant tissue and are said to be inducible. Examples include ethylene inducible promoters such as an E8 promoter, an E4 promoter (Deikman, et al., 1992; Xu et al. 1996; Deikman et al., 1988) and an E8::E4 promoter (described in co-owned U.S. Ser. No. 09/157,077, expressly incorporated by reference herein and in Clendennen et al., 1999); a banana TRX promoter, TRX fusion promoter and a banana PEL promoter (described in co-owned U.S. Ser. No. 09/527,972, expressly incorporated by reference herein).

As defined herein, a senescence-associated promoter refers to any promoter which directs RNA synthesis at higher levels in plant cells and tissues that are undergoing senescence, relative to the level of RNA synthesis in the same type of tissue of the same plant variety which is not undergoing senescence. It will be understood that the level of senescence-associated gene expression under the control of a given senescence-associated promoter will vary dependent upon the type of plant, environmental conditions etc., and will typically change as the senescence process progresses.

B. Senescence-Associated Promoter Isolation

Subtractive hybridization has been used to identify transcripts differentially expressed during the senescence process in Brassica. (See, e.g., Buchanan-Wollaston, 1994; Hanfrey et al., 1996; Buchanan-Wollaston and Ainsworth, 1997.) Type I metallothionein (MT1) protein and glutamine synthase (GS) transcripts have been identified as expressed in association with senescence in Brassica napus (Buchanan-Wollaston, 1994; Buchanan-Wollaston and Ainsworth, 1997).

Type I metallothionein (MT1) proteins are low molecular weight cysteine-rich metal-binding proteins, which have been associated with stress in plants.

Glutamine synthase (GS) encodes a cytoplasmic isoform of glutamine synthase, which is involved in amino acid metabolism. More specifically, GS catalyzes the synthesis of glutamine, a significant form of N storage and transport in plants. Cytosolic GS functions during both development and senescence to convert ammonium to glutamine to remobilize nitrogen from source to sink organs. Cytosolic GS is distinguished from the plastidic isoform by the absense of a plastid targeting presequence.

The present invention provides MT1 and GS senescence-associated promoters, isolated from Brassica oleracea (broccoli), and presented as SEQ ID NO: 1 and SEQ ID NO:2, respectively.

Upstream sequences containing the putative promoter regions of the MT1 and GS genes have been isolated from a linker-adapted genomic library of broccoli (Brassica oleracea).

The promoters were isolated using PromoterFinder™ DNA walking, which is a simple and general PCR-based method for cloning unknown genomic DNA adjacent a known sequence, e.g., a cDNA sequence. Primers for use in PromoterFinder DNA walking were designed for preferential amplification of upstream sequences associated with the MT1 and GS gene sequences, respectively.

A Clontech PromoterFinder™ Construction Kit (Clontech, Palo Alto, Calif.) was used to isolate the metallothionein MT1 and GS promoters, described herein, using the manufacturer's protocol, as further detailed in Example 1.

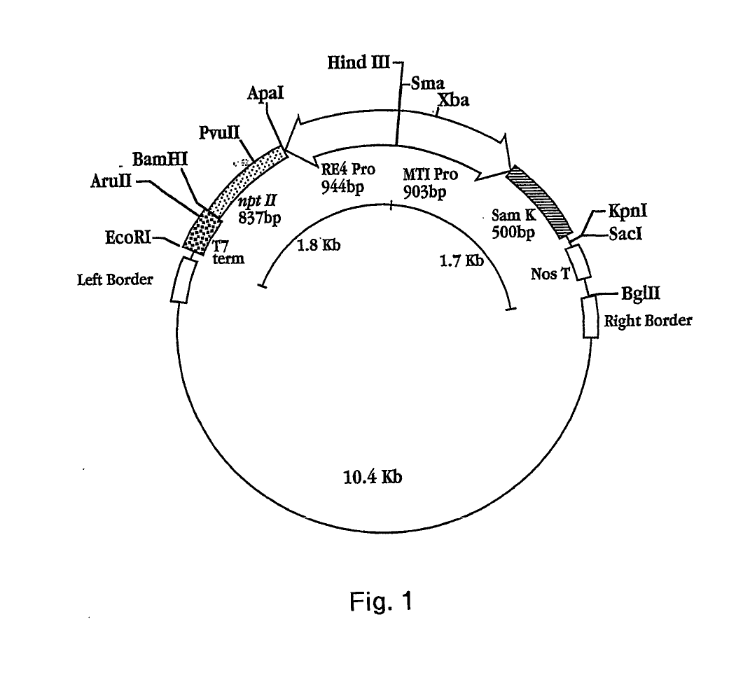

SEQ ID NO:1 comprises the complete nucleotide sequence of the senescence-associated MT1 promoter isolated from Brassica oleraceae. In SEQ ID NO:1, nucleotides 1-6 comprise the HindIII restriction site used to subclone the promoter region into pAG 4228; nucleotides 1-60 are derived from the pCR2.1 cloning vector from Invitrogen; nucleotides 61-96 represent a partial GenomeWalker Adaptor from Clontech; nucleotides 879-885 are the putative TATA box; and nucleotides 983-988 are an Nco site that was engineered around the start codon to facilitate subcloning as a translational fusion with heterologous coding regions, e.g., sam-k encoding SAMase in pAG 4228. pAG 4228 is depicted in FIG. 1.

SEQ ID NO:2 comprises the complete nucleotide sequence of the senescence-associated GS promoter isolated from Brassica oleraceae. In SEQ ID NO:2, nucleotides 1-6 represent the SmaI restriction site used to subclone the promoter region into pAG 148. Nucleotides 1-11 are from the GenomeWalker Adaptor primer (Clontech) used in the isolation of the promoter fragment. The putative TATA box is at nucleotides 2211-2217, while nucleotides 2323-2328 contain an NcoI site that was engineered around the ATG start codon to Facilitate subcloning as a translational fusion with heterologous coding regions, e.g., sam-k encoding SAMase in pAG 4248. pAG 4248 is similar to the pAG 4228 vector depicted in FIG. 1, but comprises the GS promoter in place of the MT1 promoter.

Once identified and isolated, the Brassica oleracea MT1 promoter nucleotide sequence was subjected to NCBI BLAST™ similarity search using the interface provided at http://www.ncbi.nlm.nih. gov/BLAST/ using version 2 with default parameters (open gap penalty of 11.0, extended gap penalty of 1.0, and the BLOSUM 62 matrix).

A BLASTN search of the MT1 promoter sequence indicated that the MT1 promoter sequence has identity to a 34 nucleotide segment of Brassica napus senescence-specific cDNAs encoding metallothionein (MT1) GenBank Accession Nos. S71334 and U20236, as well as weak identity to a region of the Arabidopsis genome (GenBank Accession No. AB007645).

An MT promoter fragment isolated from Brassica napus (canola) has been described (Butt et al., 1998). The regulatory region of the promoter sequence described by Butt et al. (the sequence upstream of the TATA box), does not have sequence homology to the MT1 promoter sequence of the invention. In addition, the promoter sequence described in the Butt et al. publication indicates a series of direct repeats within the upstream 1 kb of the MT promoter, while the MT1 promoter sequence of the invention reveals no direct repeats.

Once identified and isolated, the Brassica oleracea GS promoter nucleotide sequence was subjected to NCBI BLAST™ similarity search using the interface provided at http://www.ncbi.nlm.nih. gov/BLAST/ on Nov. 27, 2000 using version 2 with default parameters (open gap penalty of 11.0, extended gap penalty of 1.0, and the BLOSUM 62 matrix).

A BLASTN search of the GS promoter nucleotide sequence indicated that the GS promoter sequence has identity to mRNAs encoding cytosolic glutamine synthetase (GS) isoforms from Brassica napus (Ochs et al., 1999), and Raphanus sativus (Watanabe et al., 1994). In Brassica napus, cytosolic GS is encoded by a small gene family composed of three similar members. The region of sequence homology between the GS promoter and GS mRNAs is at the 3′ end of the GS promoter, corresponding to the 5′ untranslated region of the Brassica napus mRNAs. In addition to the GS mRNA homology, there is also a region of homology within the GS promoter to a partial coding sequence, AtPP (GenBank Accession No. AJ245480). This sequence was characterized in association with a Brassica self-incompatibility locus, and the specific region of homology was shown to be a repetitive sequence, existing in multiple copies within the Brassica napus genome. Despite the sequence homology with AtPP, there are no major open reading frames within the GS promoter, and there is no evidence to suggest that the AtPP-like sequence within the GS promoter is functional.

Related sequences may also be found at GenBank Accession Nos. AF200711, which presents a 341 bp mRNA sequence for a metallothionein-like protein from Brassica oleracea cultivar Green king; U20236, which presents a 416 bp mRNA sequence for a metallothionein-like protein from Brassica napus; and S71334, which presents a 422 bp mRNA sequence for a senescence-associated gene from Brassica napus, cv Falcon, leaves.

The present invention provides the entire sequence for MT1 and GS promoters isolated from Brassica oleracea. The promoter sequences are provided herein as nucleotides 97 to 987 of FIG. 1 and nucleotides 12 to 2327 of FIGS. 2A and 2B for the MT1 and GS promoters (SEQ ID NO:1 and SEQ ID NO:2, respectively).

C. Variant and Modified Senescence-Associated Promoter Sequences

While an entire MT1 or GS promoter sequence is sufficient for senescence-associated promoter activity, shorter or truncated MT1 or GS promoter sequences may function to direct senescence-associated gene expression.

Accordingly, the invention further provides nucleic acid sequences sufficiently homologous to the MT1 and GS promoter sequence presented herein as SEQ ID NO: 1 and SEQ ID NO:2, to be capable of selectively expressing genes in senescing tissue. One of ordinary skill in the art can appreciate that 5′ or 3′ truncations and/or internal deletions of the MT1 and/or GS sequences presented herein can be made, and those truncated forms tested for senescence-associated promoter activity. Accordingly, it will be understood that the invention includes truncated versions of the MT1 and GS senescence-associated promoters, provided herein. Such truncated MT1 and GS senescence-associated promoters are sufficient to promote gene expression in a senescence-associated manner. The exact sequence of truncated MT1 and GS senescence-associated promoters can readily be determined by truncation of the sequences presented herein as SEQ ID NO:1 and SEQ ID NO:2, followed by testing of such truncated forms for senescence-associated promotion of gene expression, as further described below.

Furthermore, sequence variants of the MT1 and GS promoters of the invention are likely to exist due to normal allelic variations. Hence, minor nucleotide additions, deletions, and mutations which do not affect the function of an MT1 or GS promoter are included within the scope of the invention. Therefore, an MT1 or GS promoter sequence may vary somewhat from the sequences presented as SEQ ID NO:1 and SEQ ID NO:2, respectively.

If an MT1 or GS promoter sequence is not identical to the sequence presented as SEQ ID NO:1 or SEQ ID NO:2, it must be sufficiently homologous to an MT1 or GS promoter sequence, respectively, as provided herein, to exhibit senescence-associated promoter activity. In other words a sufficiently homologous MT1 or GS promoter sequence must be able to promote the senescence-associated expression of a transgene operably linked thereto. “Sufficiently homologous” generally means that the promoter sequence is at least 90% homologous and preferably 95% homologous to an MT1 or GS promoter sequence, or a fragment thereof, respectively. Alternatively, a candidate promoter sequence will hybridize under moderate to high stringency conditions to an MT1 or GS promoter sequence or a fragment thereof. The MT1 and GS senescence-associated promoters presented herein and/or sufficiently homologous variants thereof may also be included as a segment of a fusion promoter for use in senescence-associated gene expression.

Any structural gene of interest may be placed under the regulatory control of a senescence-associated promoter of the invention. The structural gene may encode a polypeptide or interest or other gene product. Hence, according to the present invention, heterologous nucleic acid coding sequences (genes) may be operably linked to such a senescence-associated promoter.

In one embodiment of the invention, a senescence-associated promoter of the invention is used to modulate the senescence-associated expression of a transgene in transformed cells, and thereby alter the progression to a senescence phenotype in plants comprising such cells.

IV. Transgenic Plants with a Modified Senescence Phenotype

Genetic modification of plants to manipulate the senescence process can be used to delay development of a senescence phenotype and extend postharvest life in vegetables, fruits and cut flowers.

Significant delays in fruit ripening, flower senescence, and flower abcission were observed in tomato and petunia overexpressing the mutant ethylene receptor from ethylene insensitive Arabidopsis (Wilkinson et al., 1997; Clark et al., 1999). Senescence has also been delayed in vegetative tissues of tobacco and Arabidopsis through genetic manipulation (Fan et al., 1997; Ori et al., 1999).

Ethylene biosynthesis takes place in the early stages of senescence and the ethylene biosynthetic pathway includes a biosynthetic and an autoregulatory or feedback component. In the biosynthetic pathway leading to ethylene, methionine is converted to AdoMet (also called S-adenosylmethionine or SAM), which is converted to ACC, which is converted to ethylene. These two reactions are catalyzed by ACC synthase and ethylene-forming enzyme (EFE), respectively. A bacterial enzyme, AdoMet hydrolase (AdoMetase), which is normally not present in plant tissue, hydrolyzes AdoMet to homoserine and MTA, both of which are recycled to methionine.

An enzyme encoded by the E. coli bacteriophage T3 hydrolyzes AdoMet to homoserine and 5′methylthioadenosine (MTA). The enzyme is known as AdoMet hydrolase (AdoMetase), or as S-adenosylmethionine cleaving enzyme (SAMase) and slows the production of the metabolic precursor of ethylene, ACC. (Studier, et al., 1976; Hughes, et al., 1987a; Hughes, et al., 1987b).

Stable integration and expression of SAMase in the cells of soft fruits and vegetables has resulted in reduced ethylene production. (See, e.g., Good et al., 1994; Mathews et al., 1995a; Mathews et al., 1995b; Kramer et al, 1997). A novel ethylene responsive hybrid promoter synthesized from elements of the tomato E8 and E4 promoters has been used for the expression of SAMase in cantaloupe (Clendennen et al., 1999). Transgenic plants comprising SAMase have previously been described. See, e.g., co-owned U.S. Pat. Nos. 5,859,330; 5,783,394; 5,783,393; 5,723,746; 5,589,623; 5,416,250 and 5,750,864, expressly incorporated by reference herein.

In one aspect, the senescence-associated promoters of the invention are used to modulate ethylene production in transformed cells, and thereby delay senescence of plants comprising such cells. In this aspect of the invention, transgenic plant cells containing a promoter of the present invention are grown to produce a transgenic plant bearing vegetables, fruit or flowers. Using senescence-associated promoters to control expression of SAMase in plants, it is expected that ethylene biosynthesis associated with postharvest deterioration will be reduced without interfering with normal plant growth and development.

In one exemplary embodiment of the invention, the coding sequence for a gene product which interferes with ethylene production, e.g., SAMase, operably linked to a senescence-associated promoter of the invention is introduced into the genome of a Brassica plant using a standard Agrobacterium binary vector. The expression of SAMase upon entry into senescence results in a transgenic plant that exhibits delayed senescence. Such a plant may have an extended period of vegetative growth and may produce more flowers, seeds, vegetables and/or fruit, than a corresponding non-transgenic plant. Exemplary Brassica spp. include, but are not limited to broccoli (Brassica oleraceae), mustard (Brassica negra), bok choy (Brassica chinensis), kale (Brassica oleracea L.), cauliflower (Brassica oleracea, Botytis Group), brussel sprouts (Brassica oleracea, Gemmifera Group), kohlrabi (Brassica oleracea, Gongylodes Group), rape (Brassica napui L.), swede (Brassica napus L.), turnip or turnip hybrids (Brassica rapa L), cabbage (Brassica oleracea), chinese cabbage (Brassica campesteris).

It will be appreciated that other coding sequences which interfere with ethylene production and/or senescence may be operably linked to a senescence-associated promoter of the invention, and used to transform plant cells which may be grown to produce transgenic plants having a modified senescence phenotype. In particular, plant cells may be transformed with a heterologous nucleic acid construct comprising a senescence-associated promoter of the invention operably linked to the coding sequence for a product capable of reducing ethylene biosynthesis when expressed in plant cells, for example, S-adenosyl-methionine hydrolase (SAMase, Ferro et al., 1995; Hughes et al., 1987), aminocyclopropane-1-carboxylic acid (ACC) deaminase, a sequence antisense to ACC oxidase, a sequence antisense to ACC synthase, an ACC oxidase cosuppression molecule, or an ACC synthase cosuppression molecule, which is under the control of a senescence associated promoter of the invention. This approach may be applied to any type of plant in which the senescence associated promoters of the invention are operative.

V. Vectors for Transforming Plant Cells

The present invention further provides vectors (or heterologous nucleic acid constructs) suitable for the transformation of plants and useful for the expression of heterologous genes (transgenes). The invention further provides transgenic plant cells, transgenic plants, transgenic vegetables, fruit and flowers, carrying a senescence-associated promoter of the invention.

The senescence-associated promoters of the invention find utility in the construction of heterologous nucleic acid constructs for the senescence-associated expression of heterologous genes operably linked to the promoters. The methods and results described herein are directed to gene expression under the control of the senescence-associated promoters of the invention in transgenic plant cells. Using known, routine DNA manipulation techniques, heterologous nucleic acid constructs for expression in plants are made whereby a heterologous DNA sequence encoding a gene product of interest, is placed under the regulatory control of an MT1 or GS senescence-associated promoter of the invention. In a preferred embodiment, the vector comprises a senescence-associated promoter operably linked to a nucleic acid coding sequence and/or control sequences recognized by a plant cell. As used herein, sequences recognized by a plant cell encompass coding sequences that the plant cell is capable of expressing and control sequences that are active in regulating gene expression. Such sequences may derive from a variety of sources, including plants and other eucaryotic species, bacteria, Agrobacteria, virus, phage, etc.

The construction of vectors for use in practicing the present invention are generally known to those of skill in the art. See generally, Maniatis, et al., MOLECULAR CLONING: A LABORATORY MANUAL, 2d Edition (1989); Ausubel, FEM., et al., Eds., CURRENT PROTOCOLS IN MOLECULAR BIOLOGY, John Wiley & Sons, Inc., Copyright (c)1987, 1988, 1989, 1990, 1993 by Current Protocols; Sambrook et al (1989); Elvin, S. B., et al., Eds. PLANT MOLECULAR BIOLOGY MANUAL (1990); Houck and Pear, 1990, and Becker, et al, 1992, all of which are expressly incorporated by reference, herein.

Such expression vectors may have single or multiple transcription termination signals at the 3′ end of the DNA sequence being expressed; The expression cassette may also include, for example, (i) a DNA sequence encoding a leader peptide (e.g., to allow secretion or vacuolar targeting), (ii) translation termination signals, (iii) selectable marker genes for use in plant cells, (iv) sequences that allow for selection and propagation in a secondary host, such as an origin of replication and a selectable marker sequence.

Selectable marker genes encode a polypeptide that permits selection of transformed plant cells containing the gene by rendering the cells resistant to an amount of an antibiotic that would be toxic to non-transformed plant cells. Exemplary selectable marker genes include the neomycin phosphotransferase (nptII) gene, the hygromycin phosphotransferase (hpt) gene, the bromoxynil-specific nitrilase (bxn) gene, the phosphinothricin acetyltransferase enzyme (BAR) gene and the spectinomycin resistance (spt) gene, wherein the selective agent is kanamycin, hygromycin, geneticin, the herbicide glufosinate-ammonium (“Basta”) or spectinomycin, respectively.

The particular marker gene employed is one which allows for selection of transformed cells as compared to cells lacking the DNA which has been introduced. Preferably, the selectable marker gene is one which facilitates selection at the tissue culture stage, e.g., a kanamycin, hygromycin or ampicillin resistance gene.

In one preferred embodiment, the methods of the invention are carried out using a vector carrying the kanamycin resistance gene.

In another preferred embodiment, the methods of the invention are carried out using a vector which includes the bar gene from Streptomyces, which encodes phosphinothricin acetyl transferase (PAT), that inactivates the active ingredient in the herbicide bialaphos, phosphinothricin (PPT). PPT inhibits glutamine synthetase, causing rapid accumulation of ammonia and cell death. Transgenic plants containing this gene exhibit tolerance to the herbicide, “BASTA”. This gene can also be used as a selectable marker gene, since explants carrying the bar gene are capable of growing on selective media containing phosphinothricin (PPT), which is an active component of bialaphos.

In further embodiments, the methods of the invention are carried out using a vector which includes an herbicide resistance gene, conferring resistance to glyphosate-containing herbicides. Glyphosate refers to N-phosphonomethyl glycine, in either its acidic or anionic forms. Herbicides containing this active ingredient include “ROUNDUP” and “GLEAN”. Exemplary genes for imparting glyphosate resistance include an EPPSP synthase gene (5-enolpyruvyl-3-phosphoshikimate synthase) or an acetolactate synthase gene.

The selection of an appropriate promoter effective to express the selectable marker-encoding sequence and the termination element for the selectable marker-encoding sequence may be accomplished by the use of well known, and/or commercially available sequences.

Typical secondary hosts include bacteria and yeast. In one embodiment, the secondary host is Escherichia coli, the origin of replication is a colE1-type, and the selectable marker is a gene encoding ampicillin resistance. Origin of replication and selectable marker sequences operative in secondary hosts are well known in the art and many are commercially available (e.g., Clontech, Palo Alto, Calif.; Stratagene, La Jolla, Calif.).

The vectors of the present invention are useful for senescence-associated expression of nucleic acid coding sequences in plant cells for use in producing a transgenic plant, which has a modified senescence phenotype. For example, a selected peptide or polypeptide coding sequence is inserted into an expression vector of the invention operably linked to an MT1 or GS promoter, as described herein. The vector is then transformed into progenitor plant cells, which are cultured under conditions effective to regenerate a plant, allowing for the expression of the protein coding sequence in the cells of the plant.

An exemplary heterologous nucleic acid construct is a standard Agrobacterium binary vector containing a DNA sequence encoding a gene product effective to modify a phenotypic characteristic of the plant, e.g., to reduce ethylene biosynthesis in vegetables, fruit and/or flower producing plants, operably linked to a senescence-associated promoter of the invention, such that transgene expression is associated with the onset of senescence. The DNA sequence is heterologous to the promoter and the heterologous nucleic acid construct contains the appropriate regulatory elements necessary for expression in a plant cell.

Standard Agrobacterium binary vectors are known to those of skill in the art and many are commercially available, an example of which is pBI121 (Clontech Laboratories, Palo Alto, Calif.), as further described, above. Preparation of Agrobacterium tumefaciens cultures is carried out using methods generally known in the art.

Constructs have been generated that place SAMase under the control of each of the MT1 and GS promoters described herein, wherein the constructs include the nptII selectable marker expressed under the control of the RE4 promoter. (See Example 2.) It will be understood that the vectors described herein may form part of a plant transformation kit. Other components of the kit may include, but are not limited to, reagents useful for plant cell transformation.

VI. Methods for Transforming Plant Cells

A heterologous nucleic acid construct containing a senescence-associated promoter of the invention, e.g., a MT1 or GS promoter, may be transferred to plant cells by any of a number of plant transformation methodologies, including Agrobacterium-mediated transformation, electroporation, microinjection, microprojectile bombardment calcium-phosphate-DNA co-precipitation or liposome-mediated transformation. (See, e.g., Comai and Coning, 1993; Klein, et al., 1988; Miki, et al. 1987; Bellini, et al., 1989).

In one preferred embodiment, a heterologous nucleic acid construct is introduced into a plant by way of a T-DNA-less Ti plasmid carried by Agrobacterium tumefaciens, followed by co-cultivation of the A. tumefaciens cells with plant cells. In such cases, vectors for use in the invention contain a selectable marker gene, T-DNA border regions from Agrobacterium tumefaciens, a heterologous gene of interest, and other elements as desired. Exemplary Agrobacterium transformation vectors are commercially available from Clontech (Palo Alto, Calif.) and further described by An, et al., 1985.

Other suitable heterologous nucleic acid constructs may be constructed using the promoters of the present invention and standard plant transformation vectors, which are available both commercially (Clontech, Palo Alto, Calif.) and from academic sources [Salk Institute, Plant Biology Labs; Texas A & M University; Waksman Institute, Rutgers, The State University of New Jersey, Piscataway, N.J.].

DNA may be introduced into plant cells by microprojectile bombardment using microparticles loaded with DNA which are bombarded into the cells using “gene gun” technology. (See, e.g., Robinson, H L and Torres, Calif., 1997.) Transformed plant cells obtained as a result of transformation with a heterologous nucleic acid construct comprising a senescence-associated promoter of the invention are cultured in medium containing the appropriate selection agent to identify and select for plant cells which express the heterologous nucleic acid sequence. After plant cells that express the heterologous nucleic acid sequence are selected, whole plants are regenerated from the selected transgenic plant cells. Techniques for regenerating whole plants from transformed plant cells are generally known in the art.

In one aspect, preferred plants suitable for transformation using the senescence-associated promoters of the invention include but are not limited to Brassica species, including, but not limited to broccoli (Brassica oleraceae), mustard (Brassica negra), bok choy (Brassica chinensis), kale (Brassica oleracea L.), cauliflower (Brassica oleracea, Botrytis Group), brussel sprouts (Brassica oleracea, Gemiifera Group), kohlrabi (Brassica oleracea, Gongylodes Group), rape (Brassica napus L.), swede (Brassica napus L.), turnip or turnip hybrids (Brassica rapa L), cabbage (Brassica oleracea), chinese cabbage (Brassica campesteris).

In another aspect, preferred plants suitable for transformation using the senescence-associated promoters of the invention include but are not limited to non-Brassica vegetable crops such as sugar beet (Beta vulgaris), green bean (Phaseolus vulgaris), celery (Apium graveolens), chard (Beta vulgaris), cucumber (Cucumis sativus), eggplant (Solanum), peppers (Capsicum), pumpkin (Cucurbita), rhubarb (Rheum), winter squash (Cucurbita), summer squash (Cucurbita pepo), zucchini (Cucurbita pepo), lettuce (Lactuca), radish (Raphanus), carrot (Daucus carota), pea (garden) (Pisum sativum), potato (Solanum), tomato (Lycopersicon) and corn (Zea mays).

In a further aspect, preferred plants suitable for transformation using the senescence-associated promoters of the invention include but are not limited to (1) fruits such as tomato (Lycopersicon); grape (Vitis); strawberry (Fragaria); raspberry, blackberry, loganberry (Rubus); currants and gooseberry (Ribes); blueberry, bilberry, whortleberry, cranberry (Vaccinium); kiwifruit and Chinese gooseberry (Actinida); apple (Malus); pear (Pyrus); melons (Cucumis sp.); members of the Prunus genera, e.g. plum, cherry, nectarine and peach; sapota (Manilkara zapotilla), banana (Musa), mango (Mangifera indica), papaya (Carica papaya), avocado (Persea); apricot (Armeniaca vulgaris); peach (Amygdalus Persia); pineapple (Nanas colossus); passion fruit (Passiflora edulis), citrus (Citrus spp); date palm (Phoenix dactylifera); plantain (Musa spp.); kiwi fruit (Actinidia chinensis) and fig (Ficus carica); (2) grains such as soybean (Glycine max), rice (Oryza), maize (Zea mays), wheat (Triticum), barley (Hordeum) and oat (Avena); (3) oil-producing plants such sesame (Sesamum indicum), safflower (Carthamus tinctorius), sunflower (Helianthus annuus), peanut (Arachis hypogaea), palm (Erythea salvadorensis), oil palm (Elaeis guineensis), olive (Olea europaea), rape or canola (Brassica napus or Brassica rapa), coconut (Cocos nucifera), corn (Zea mays), soy bean (Glycine max) and other oilseed crops; (4) nut-producing plants, such as pistachio (Pistacia vera), walnut (Juglans regis), cashew-nut (Anacardium occidentale), rape (Brassica napus), pecan-nut (Carya illinoensis), macadamia nut (Macadmia terniflora), brazil nut (Bertholletia excelsa), and almond (Prunus communis); (5) plants used to produce cut flowers such as carnation (Dianthus), iris (Iris), rose (Rosa), daisy (Asteraceae), lily (Lilium), snapdragon (Antirrhinum) and petunia (Petunia); and (6) other crops such as cotton (Gossypium), alfalfa (Medicago sativa), turfgrass (Poaceae family), flax (Linum usitatissimum), and tobacco (Nicotiana).

Optimal methods for plant transformation vary dependent upon the type of plant. For example, in soybean one preferred method for Agrobacterium-mediated transformation requires removal of the hypocotyl tissue (U.S. Pat. Nos. 5,824,877 and 5,569,834) and in tomato, a method for transformation of hypocotyl explants relies on the use of feeder cells or nurse cultures, as described in Frary A, and Earle ED, 1996.

Several varieties of Brassica oleraceae have been successfully transformed using Agrobacterium tumefaciens and A. rhizogenes strains, as reviewed in Puddephat et al., 1996. The most widely used explant for plant regeneration and transformation in Brassica species is hypocotyl taken from seedling tissue. (See, e.g., Maloney, M. M. et al., 1989). See, also U.S. Pat. Nos. 5,750,871 and 5,463,174, which are directed to methods involving tobacco feeder cells that act as a nurse culture for Brassica explants.

Transgenic Brassica plants have also been recovered using explants such as cotyledon, internode, leaf, peduncle, petiole, protoplasts from leaf mesophyll tissue, seedling (hypocotyl and cotyledonary petiole) and stem.

As detailed in Example 3, heterologus nucleic acid constructs containing the coding sequences for sank or GUS were introduced into both cauliflower (Brassica olearaceae L. var. Botrytis) and broccoli (Brassica olearaceae L. var. Italica) using Agrobacterium tumefaciens. In cauliflower, transgenic plants were generated by co-cultivation of stem, petiole and hypocotyl explants. In broccoli, although hypocotyl segments resulted in a high rate of shoot regeneration, repeated attempts at transformation using hypocotyl explants yielded non-regenerable calli or untransformed shoots. In an alternative approach, shoot tip halves were used as source explants and transformed broccoli plants containing a samk or GUS transgene along with an antibiotic resistance gene, nptII or hpt, were successfully recovered.

In one exemplary embodiment of the invention, shoot tip segments of a commercial broccoli variety of Brassica oleraceae are transformed according to known methods using a disarmed Agrobacterium strain to introduce selected binary vectors into plants. (See, e.g., Hosoki T et al., 1991; Cough, S J and Bent, A F, 1998.) In practicing this embodiment of the method, floral tissues are dipped into a solution containing a disarmed strain of Agrobacterium tumefaciens, 5% sucrose and a surfactant Silwet L and primary transformants are selected on the basis of their capacity to regenerate and develop roots on media containing a selective agent.

In a further exemplary embodiment of the invention, a hypocotyl or shoot tip transformation method which does not require the use of feeder cells or nurse cultures is employed to introduce Agrobacterium vectors into plant cells.

Although Brassica plants are exemplified herein, a heterologous nucleic acid construct comprising a senescence-associated promoter of the invention may be used to effect the senescence-associated expression of a transgene in any type of plant.

Accordingly, in further exemplary embodiments, any of a number of Agrobacterium transformation methods may be used to transform plant cells using the senescence-associated promoters of the invention. Agrobacterium transformation has been previously described for a large number of plants, including: rice, tomato, apple, almond, asparagus, avocado, broccoli, carrot, cauliflower, celery, cucumber, grape, persimmon, and spinach. See, e.g., Sagi et al., 1995 (banana); Ranier et al., 1990 (rice); McCormick et al., 1986 (tomato), Van Eck J M, et al., 1995 (tomato); Norelli et al., 1996 (apple); Miguel C M et al., 1999 (almond); Cabrera-Ponce J L et al., 1997 and Delbreil B et al., 1993 (asparagus); Mogilner N et al., 1993 (avocado); Hosoki T et al., 1991 (broccoli); Hardegger M et al., 1998 (carrot); Bhalla P and Smith N, 1998 (cauliflower); Catlin D et al., 1988 (celery); Sarmento G G et al., 1992 and Trulson A J et al., 1986 (cucumber); Scorza R et al., 1995 and Franks T et al., 1998 (grape); Nakamura Y et al., 1998 (persimmon); and Zhang H X and Zeevaart J A D, 1999 (spinach).

VII. Identification and Evaluation of Gene Expression in Transgenic Plant Cells and Plants

Following transformation, transgenic plant cells are assayed for expression of a transgene which is operably linked to a senescence-associated promoter of the invention. Transgenic plant cells may be initially selected by their ability to grow in the presence of a selective agent, such as the aminoglycoside antibiotic, kanamycin.

Expression of a transgene may also be determined by analysis of DNA, mRNA, and protein, associated with the expression of the transgene. The assays are typically conducted using various plant tissue sources, e.g., vegetables, fruit, flowers, leaves or stems.

Transgene expression may be measured by analyzing a sample directly, for example, by conventional Southern blotting; Northern blotting to quantitate the transcription of mRNA, dot blotting (DNA or RNA analysis), RT-PCR (reverse transcriptase polymerase chain reaction) to confirm expression of the transgene, or in situ hybridization, using an appropriately labeled probe, based on the sequence of the particular transgene being expressed.

Alternatively, gene expression may be measured by immunological methods, such as immunohistochemical staining of cells or tissue sections to directly evaluate expression of the transgene. Antibodies useful for immunohistochemical staining and/or assay of sample fluids may be either monoclonal or polyclonal, and may be prepared in any mammal. Exemplary methods are described below.

In another approach, putative senescence-associated promoter elements can be verified by cloning into a reporter vector system exemplified by GUS (β-glucuronidase), and assaying for promoter function by evaluating expression of the reporter gene in a transient assay system using a reporter gene, effective to evaluate the senescence-associated expression under the control of an MT1 or GS promoter. Expression of GUS protein is easily measured by fluorometric, spectrophotometric or histochemical assays (Jefferson, 1987a).

A. DNA PCR and Southern Blot.

DNA may be extracted from various tissues of transgenic plants and analyzed for the presence of the transgene coding sequence by polymerase chain reaction (PCR), using procedures routinely employed by those of skill in the art. PCR may be carried out using oligonucleotide primers specific to the transgene coding sequence.

Transformation of each plant may be confirmed by carrying out a Southern blot analysis of genomic DNA. Typically, total DNA is isolated from each transformant (e.g., Schwarz-Sommer, et al., 1992). The DNA is then digested with restriction enzyme, fractionated in an agarose gel and transferred to nitrocellulose filters (e.g., HYBOND-N, Amersham) or nylon membranes, according to standard techniques. The blot is then probed, e.g., with ³²P-labeled cDNA. Procedures for restriction digestion, gel electrophoresis, Southern transfer and hybridization are as described by Maniatis et al., 1989, expressly incorporated by reference herein.

A polymerase chain reaction (PCR) reaction mixture generally includes the components necessary for amplification of specific nucleic acid sequences. Kits and reagents for carrying out PCR reactions are commercially available. For example, a Taqman™ (PE Applied Biosystems) probe and primer set may be designed and used for the detection of the transgene nucleic acid sequence.

B. Northern Blot

RNA may be isolated from specific plant tissues and separated, e.g., in a 1.2% agarose gel containing 2.2M formaldehyde, then blotted to a nylon filter, e.g., Hybond-N, according to the standard procedures routinely used in the art. Strand specific RNA probes may be synthesized by phage T7 and T3 RNA polymerases from a cDNA clone for the transgene. This allows for a determination of the presence and an estimation of the amount of mRNA resulting from expression of the transgene at various stages of the development of the plant, e.g., prior to and following the onset of senescence in the plant. Northern analysis may be carried out as described in Maniatis et al., 1989.

C. RT-PCR

RNA may also be extracted from various plant tissues, followed by reverse transcription of mRNA and amplification of partial cDNA sequences using polymerase chain reaction (PCR) and methods generally known in the art together with reagents which commercially available (e.g., from Perkin Elmer). RT-PCR may also be carried out at various stages of the development of the plant, e.g., prior to and following the onset of senescence.

D. Immunoassay or Western Blot

An ELISA, Western blot or immunodotblot immunoassay may be conducted on putative transformants to detect the presence of the protein encoded by the transgene, as generally described in Harlow and Lane, 1988. Standard techniques for Western blotting are known in the art, e.g., the protocol described in Glick, B. R. and Thompson, J. E., Eds. METHODS IN PLANT-MOLECULAR BIOLOGY AND BIOTECHNOLOGY, p. 213-221, CRC Press, 1993.

In addition, given an antibody that is specifically immunoreactive with a protein or polypeptide encoded by the transgene, any of a number of different types of immunoassays may be employed by one of skill in the art to detect the presence of the encoded protein in tissues of transgenic plants.

E. Evaluation of Promoter Activity Using Reporter Constructs

The relative activity of the senescence-associated MT1 and GS promoters of the invention may be determined in a transient assay system using a reporter gene, which is effective to evaluate the senescence-associated expression from the promoters. Expression of a reporter gene, e.g., GUS (β-glucuronidase) is easily measured by fluorometric, spectrophotometric or histochemical assays (Jefferson, 1987).

F. Evaluation of Gene Expression in Transgenic Plants

When a senescence-associated promoter of the invention is used to control the expression of a transgene product associated with ethylene production, gene expression may be evaluated by observing the transgenic plant relative to non-transgenic plants of the same variety grown under the same conditions. The morphological changes typically associated with ethylene production in seedlings generally include the “triple response”. The morphological changes typically associated with ethylene production in leaves generally include abcission, chlorosis and dehydration. The morphological changes typically associated with ethylene production in fruit generally include dehydration, loss of pigments and flavor components, as well as softening and cell wall degradation leading to pathogen susceptibility.

The morphological changes typically associated with ethylene production in vegetables generally include dehydration, softening, wilting and browning. The morphological changes typically associated with ethylene production in flowers generally include petal collapse and abcission, browning and stem wilting. The effect of transgene expression under the control of the senescence-assoicaited promteors of the invention may be evaluated based on the above-refenced morphological changes associated with ethylene production using methods generally known in the art.

VIII. Utility

Interfering with the senescence process is an alternative approach to the direct modulation of ethylene biosynthesis and/or modulation of ethylene regulation following biosynthesis in order to extend the postharvest life of vegetables, fruits and flowers. The present invention is directed to senescence-associated promoters which may be used to express a transgene in plants when the plant is undergoing senescence.

The invention provides the MT1 and GS senescence-associated promoters presented herein as SEQ ID NO:1 and SEQ ID NO:2, respectively, together with homologues, variants, truncated forms and fusions thereof, which exhibit the ability to promote the senescence-associated expression of a nucleic acid coding sequence operably linked thereto. In a related aspect, the invention provides vectors or heterologous nucleic acid constructs, plant cells and plants comprising such senescence-associated promoters, homologues, variants, truncated forms or fusions thereof.

The invention finds utility in the generation transgenic plants that exhibit delayed senescence and methods of developing such plants. More specifically, the senescence-associated expression of a transgene provides a means for delaying post-harvest senescence in plants and associated quality deterioration in vegetables, fruits and flowers. Such temporal gene expression finds utility as an effective strategy to interfere with the deleterious effects of senescence-associated ethylene production on vegetables, fruits and flowers.

Although the invention has been described with respect to particular embodiments, it will be apparent to those skilled in the art that various changes and modifications can be made without departing from the invention.

The following examples illustrate but are not intended in any way to limit the invention.

IX. Materials and Methods

Biological reagents were typically obtained from the following vendors: 5′ to 3′ Prime, Boulder, Colo.; New England Biolabs, Beverly, Mass.; Gibco/BRL, Gaithersburg, Md.; Promega, Madison, Wis.; Clontech, Palo Alto, Calif.; and Operon, Alameda, Calif.

Standard recombinant DNA techniques were employed in all constructions. See, e.g., Adams and Yang, 1977; Ausubel, et al., 1992; Hooykaas and Schilperoot 1985; Sambrook, et al., 1989, expressly incorporated by reference herein.

All publications, patents and patent applications are herein expressly incorporated by reference in their entirety.

### EXAMPLE 1

The present invention includes the isolation and characterization of an upstream region associated with the Brassica oleraceae (broccoli) MT1 and GS genes. The isolated MT1 promoter fragment is approximately 988 bp in length and the isolated GS promoter fragment is approximately 2328 bp in length. Nucleotides 97 to 987 of SEQ ID NO:1 represent the complete nucleotide sequence of the MT1 promoter, and nucleotides 12 to 2327 of SEQ ID NO:2 represent tie complete nucleotide sequence of the GS promoter.

A. Isolation of MT1 and GS Promoter Fragments

A CTAB (hexadecyltrimethylammonium bromide) extraction buffer was used for extracting genomic DNA. (See, e.g., Stewart, C N Jr., and Via LE, BioTechniques, 14(5):748-749, 1993.)

DNA was resuspended in TE buffer (10 mM Tris HCl, pH 7.4, 1 mM EDTA), containing RNase, incubated at 55° C. for 15 minutes, further extracted phenol/chloroform, then chloroform, and PCR was used to amplify the glutamine synthetase genomic (GS) fragment and the metallothionein (MT1) genomic fragment using primers that were designed based on Brassica napus (rapeseed) cDNA sequences found at GenBank Accession #U20236 was used for MT1 and GenBank Accession #X76736 for GS, respectively.

PCR was carried out PCR using a RoboCycler® under the standard conditions: 1 cycle (97 C, 3 minutes); 2 cycles (97 C, 1 minute), then 0.5 μl AmpiTaq 5U/μl was added followed by 57 C for 1 minute and 72 C for 1 minute; then 25 cycles (94 C, 1 minute; 57 C, 1 minute; 72 C, 1 minute); and 72° C. for 7 nm then 6 C for 99 minutes.

A 5 μl aliquot of each amplification reaction was separated by agarose gel electrophoresis, the gels were stained and analyzed. PCR amplification using the GS- and MT1-specific primers yielded PCR products of approximately 1.8 kb and 300 bp, respectively. These fragments were used in the design of primers for use in DNA walking to identify and isolate the full length promoter sequences as set forth below.

B. Promoter Finder Libraries

Using genomic DNA, upstream sequences containing the putative promoter regions of MTI and GS genes were isolated from a linker-adapted genomic library of broccoli (Brassica olearacea). The promoters were isolated using the Universal Genome Walker Kit (previously called PromoterFinder™ from Clontech Laboratories, Inc., Palo Alto, Calif.), a simple and general PCR-based method for cloning unknown genomic DNA adjacent to a known sequence.

The libraries were constructed and screened according to the supplier's protocol.

Briefly, broccoli genomic DNA was digested with the five restriction endonucleases included in the kit (DraI, EcoRV, PvuII, Sca I and StuI). Promoter Finder Adapters were ligated to the digested genomic DNA fragments, and a combination of adapter and gene-specific oligonucleotide primers were used in nested PCR reactions to walk up or downstream in the genome. Primers for use in the Genome Walker walking were designed for preferential amplification of upstream sequences associated with the MT1 and GS gene sequences. PCR was carried out using a RoboCycler® under the following conditions: 7 cycles (94 C, 25 seconds, 72 C, 3 minutes); 32 cycles (94 C, 25 seconds, 67 C, 3 minutes); 72° C. for 7 min and 25 C for 60 minutes).

An approximately 1000 bp product was amplified from a Pvu II library after two rounds of amplification of the broccoli genomic libraries using MT1-specific and adapter primers. An approximately 2500 bp product was amplified from the Pvu II library after two rounds of amplification of the broccoli genomic libraries using GS-specific and adapter primers. Based on the size and sequence of the amplification products, it was determined that there was sufficient sequence information to use the amplification fragments to directly subclone the MT1 promoter and the GS promoter from the amplification products.

A DNA fragment containing the upstream regulatory region for each of the senescence-associated genes was amplified directly from the cloned GenomeWalker products. For ease of cloning, an Nco site was engineered around the start codon in the 3′ primer designed for each promoter. The sequences contained putative TATA boxes and had high A/T%, characteristic of plant regulatory regions.

### EXAMPLE 2

Preparation of pAG 4248 (CS/SAMase) and pAG 4228 (MT1/SAMase) Construct

Recombinant nucleic acid constructs comprising the GS and MT-1 senescence associated promoters of the invention and the sam-K gene are prepared using the modified promoter sequences described in Example 1 and techniques routinely employed by those of skill in the art.

In a typical construction, the promoter sequences were digested to produce the appropriate cohesive ends, using Hind m and Nco for MT1, and Sma and Nco for GS, then cloned into compatible sites in a heterologous expression construct, comprised of the promoter translationally fused with samk. The resulting constructs were named pAG128 and pAG148, containign MT1 and GS poromoters, respectively. The nucleotide sequence of the two promoters, as they exist in pAG128 and pAG148, are presented in SEQ II) NOs:1 and 2, respectively.

### EXAMPLE 3

Generation of Transgenic Plants in Broccoli (Brassica Olearceae)

Several varieties of Brassica oleraceae have been successfully transformed using Agrobacterium tumefaciens, with hypocotyl the most widely used explant for plant regeneration and transformation in Brassica species. (See, e.g., as reviewed in Puddephat et al., 1996: and Maloney, M. M. et al., 1989).

Heterologus nucleic acid constructs containing the coding sequences for samk, nptII, hpt or GUS were introduced into both cauliflower (Brassica olearaceae L. var. Botrytis) and broccoli (Brassica olearaceae L. var. Italica) using Agrobacterium tumefaciens. Transgenic plants were grown to maturity and stable integration of the genes were confirmed by molecular methods and progeny analysis.

In cauliflower, transgenic plants were generated by co-cultivation of stem, petiole and hypocotyl explants.

In broccoli, although hypocotyl segments gave a shoot regeneration rate of 84 to 100%, repeated attempts at transformation using hypocotyl explants yielded non-regenerable calli or untransformed shoots. In an alternative approach, shoot tip halves were used as source explants and transformed broccoli plants containing a sam or GUS transgene along with an antibiotic resistance gene, nptII or hpt, were successfully recovered.

Single well developed colonies of Agrobacterium tumefaciens strains EHA101/105 containing desired binary plasmids, were transferred to 20 ml of MGL medium, supplemented with 5014M acetosyringone at pH 5.6. This was grown overnight (16-18 hr) and used in co-cultivation.

Hypocotyl and shoot tip half segments (longitudinal sections) from two week old broccoli (Brassica olearaceae L. var. Italica) seedlings were used for transformation. The full length hypocotyls were cut out, streaked with a pointed scalpel blade (#11) and immediately placed in an Agrobacterium suspension. 3 to 5 mm shoot tips were halved lengthwise and placed in a Agrobacterium bacterial suspension. The Agrobacterium incubated explants were transferred to 125 ml flasks containing 25 ml solid or liquid medium (MS salts, B5 vitamins, sucrose 3%, 1 mg/l 2, i-P, 50 μM acetosyringone at pH 5.8, with solid medium also containing 0.2% phytagel).

Approximately 10 hypocotyls and shoot tip segments were cultured per petri plate (solid medium) or in flasks containing liquid medium under agitation at 100 rpm. In the cases of liquid medium treatment, the solution was decanted, replaced with fresh medium on day 2 and on day 3, the medium was decanted and explants were rinsed with MS liquid medium fortified with cefotaxime at 500-1000 mg/1 (depending on the turbidity of suspension medium).

Following the rinsing step, the explants were placed on selection medium containing 500 mg/l carbenicillin and 5 mg/l geneticin or hygromycin. Cultures were observed at regular intervals of approximately 2 to 3 weeks and explants with callus and/or shoot primordia and/or shoots, were transferred to fresh medium. The concentration of the selection agent was gradually increased up to 15 to 20 mg/l geneticin or hygromycin. The tissues that were sensitive to antibiotic (evidenced by bleaching and/or browning) were discarded at each transfer step. The green or partially green shoots from shoot tip explants were excised and subjected to repeated segmenting (longitudinal sectioning) before subculture to fresh medium for a period of 4 months until the tissues were insensitive to antibiotic. Well developed shoots are transferred to rooting medium and rooted plants were confirmed for stable integration of the transgene by Southern analysis.

Table 1 reflects the results of Agrobacterium-mediated transformation of hypocotyl and shoot tip tissue of broccoli (Brassica oleraceae) uaing 4 different vectors: pAG1401 (noshpt, 35Sgus), containign the nos terminator (nos), the hpt selectable marker and GUS under the control of the CaMV 35S promoter; pAG1552 (nosnptII, E4SAM), containign the nos terminator (nos), the nptII selectable marker and SAMase under the control of the tomato E4 promoter; and pAG1452 (noshpt, E4SAM), containign the nos terminator (nos), hpt selectable marker and SAMase under the control of the tomato E4 promoter.

TABLE 1
Transformation of Hypocotyl vs. Shoot tip of Broccoli (Brassica
oleraceae), (cultivar B-24-3)

| | Explant | | Transforamtion |
|---|---|---|---|
| Plasmid | Tissue | # of Explants | Frequency¹ |
| pAG1401² | Hypocotyl | 219 | 0 |
| noshpt, | Shoot tip | 106 | 1% (1) |
| 35SGus | | | |
| pAG1401³ | Hypocotyl | 240 | 0 |
| noshpt, | Shoot tip | 124 | 1% (1) |
| 35SGus | | | |
| pAG1552 | Hypocotyl | 200 | 0 |
| noshpt, | Shoot tip | 182 | 2% (3) |
| E4SAM | | | |
| pAG1452 | Hypocotyl | 200 | 0 |
| noshpt, | Shoot tip | 190 | 4% (8) |
| E4SAM | | | |

¹transformation frequency refers to the ratio of number of transgenic plants recovered to the total number of explants. Control cultures of broccoli give regeneration frequencies of 84% and 100% in hypocotyl and shoot tip explants, respectively.
²co-cultivation step was carreid out using solid media.
³co-cultivation step was carreid out using liquid media

The results show that transformation of shoot tip was sucessful with each of the vectors described above. In contrast, no transforamnts were generated when hypocotyl was used as the explant tissue.

### EXAMPLE 4

Delayed Senescence Phenotype in Brassica olearceae

We tested the ability of a transgene comprising the MT1 promoter operably linked to a senescence associated gene to effect delayed senscence in Brassica olearceae. We transformed rapidly cycling cabbage with the pAG4928 construct, in which the MT1 gene directs expression of the SAMase gene (Hughes, 1987). pAG4928 is similar to the pAG4228 vector depicted in FIG. 1 but lacks the nptII selection cassette, including the RE4 promoter and the termination element.

We obtained twelve T1 lines in which we verified the presence of the SAMase gene by PCR analysis. The senescence phenotype of there lines, as compared to the non-transformed genotype, was assesed using the “detached leaf senescence test”. We rated the degree of yellowing of detached leaves placed on moist paper at 15C in darkness after various days of incubation. We repeated this test twice, both times using two replicates per line, 15 plants per line/replicate. Seven of the twelve MT1 lines showed significantly enhanced shelf life compared to the controls. Yellowing of leaves of these seven lines lines proceeded less rapidly compared to the control line.

It is claimed:

1. An isolated nucleic acid molecule comprising a senescence-associated promoter selected from the group consisting of:
(a) an MT1 promoter comprising a nucleotide sequence presented as SEQ ID NO:1; and
(b) a promoter comprising a nucleotide sequence having at least 95% sequence identity to the sequence presented as SEQ ID NO:1 or a fragment thereof, wherein % identity is calculated using the LALIGN program found in the FASTA Version 2.0 suit of programs using default parameters with the BLOSUM50 matrix, a ktup of 2 and a gap penalty of −12/−2, where said isolated nucleic acid sequence hybridizes to the sequence presented as SEQ ID NO:1 or a fragment thereof, under high stringency conditions;
wherein the senescence-associated promoter, when operably linked to a nucleic acid encoding a gene product, promotes senescence-associated expression of the gene product.

2. A plant expression vector comprising the nucleic acid molecule of claim 1, wherein the senescence-associated promoter is operably linked to a nucleic acid encoding a gene product.

3. The plant expression vector of claim 2, wherein the gene product is associated with delayed senescence.

4. The plant expression vector of claim 3, wherein the gene product is ethylene-associated.

5. The plant expression vector of claim 4, wherein the gene product is S-adenosyl methionine hydrolase (SAMase).

6. A transgenic plant cell comprising the plant expression vector of any one of claims 2, 3, 4, and 5.

7. A transgenic plant having a delayed senescence phenotype, comprising in its genome a plant expression vector according to claim 3, wherein expression of the gene product associated with delayed senescence interferes with normal senescence of vegetables, fruit or flowers of said transgenic plant.

8. A transgenic plant according to claim 7, wherein the senescence-associated promoter comprises a nucleotide sequence of SEQ ID NO:1.

9. A transgenic plant according to claim 7 or 8, wherein the gene product associated with delayed senescence is S-adenosyl methionine hydrolase (SAMase).

10. A method of producing a transgenic plant that exhibits senescence-associated expression of a gene product, comprising:
(a) transforming plant cells with the plant expression vector of claim 2; and
(b) growing said transformed plant cells to produce a transgenic plant that exhibits senescence-associated expression of the gene product.

11. The method of claim 10, wherein expression of the gene product delays senescence.

12. The method of claim 11, wherein the gene product is S-adenosyl methionine hydrolase (SAMase).

13. The method of claim 11 or 12, wherein said transgenic plant is a Brassica plant.


SEQUENCE LISTING (2 sequences)

SEQ ID NO: 1 - DNA, 988 bp, Brassica oleracea
aagcttggtaccgagctcggatccactagtaacggccgccagtgtgctggaattcggcttactatagggcacgcgtggtcgacggcccgggctggtactataagacgtggaagaaaggatgttgtgcgtcctcttaatgtctttatcagaaggagcaacttgttagaaataatctaattagataaataattttgacaaaagaaatttagttaattagttaatgaataatagacgtgctggttgaatgaatataataatatgttataaatattcatactaggtcaaaatgaaaggtacattttctagaaaggtatagtaagcagatgaaacaggtacgtttcatgcctcttattttggttggctgtcgtaactctttgttgcgtgtacggctttttcgtattccctattaatttgacgtgctccatcaattttttgaaactcaaataaaacattagactattttcttgaaaatggtagaggaaatcttattaaaaaaaaagaaatagtgggcaagttgacgttatttcttcaaagaagaagaagaaaagtttgatgttattcttatgaaaagaagaaaacagcatataatttagtgcctatatagtaaacaaaaattatttttcctttagccactaatgatccataaaaaatgtttattttgaggcaaattaatttaacagttttaatatacgatatttttaatataagataaatataagatcctctcagtattattttaaggttataaccaaaataaatagaagaatcactacgaaatgctctatatgatagataacttattctaaaagcatttgattttatccatacacggaatgaaacgtgtatggtgtaaacaacaccgactctttctcatcttttataaataggtggtcattccctttgttcaagtcacaagacataaagcaatcaaaggaagaagaaacaaaaacaaactacaactttaaatcaaagagaagagaagccatgg

SEQ ID NO: 2 - DNA, 2328 bp, Brassica oleracea
  misc_feature (551)..(551): “n” is A, C, G, or T
cccgggctggtctgaagcttcttattttcttagtctttttcctttaatattttgctttcttttagcatcgattaaattgaagtgagggttataaatttgatggataataactagaaaaattcttcacaacttctttcttatttataaaagaacagagtttcactcgaaaataggtacatattttataactgattaaaccgaataaaccgaacaaaaccgatcaaaaatagaaagatgtaaatatgtacatactttataacgtcacatgaaaatctatttgttatataagttattttttttgttaataattaataccatatttttctatagtaataaagaatcctaatttgaaaaatacttaaaatataattaaataacaattcatcgcaactgaggcttcttattttcttagttcttcttttaatcattttgatttcttttagcattgattaaactgaagtgaatgttataaatttgatggataataactagaaaaagttcgtaacaacttttttcttatttataaacgaacatagtttcactcgaagaancatgactttgatggacactaaatatggaagaatggaaaaacttttctttcatacttctttttttgttttttgtttttatttttattttcaaaatttaaagctttgattttaattctagatttgattatttcatctgaaggtataagcgtgtttttttgttcttttatttgaaaatataatatatttttaataaatgattgcgatgacaatatgactctaaaatttatataatatgatcccacactaaataattatgttttgatttaaaaccgaataaaccgaaaacgacggtaaataaaccgaaccgaagtaaatatagatttagaatggtatttatattttactaactgaaataccgaaaacccgaaaaaaccgaacccaaaccgaaatgatatccggattgaacaccgctattaaattaatgtattttaatttcatttatttattttctattttatttaatcatttgaattttatttagttttctgatagctttacaaagaatatatggtttgtttcgtataactagattttcgacgaatcattttcaataatataatattaatttaacaaaaaaaaagtataatgtaagatgatgaaaatgaagaatattaatttatatgaaaaatgatgacgtcaaagcttttattgttggtgttttcaaaggggaagtctcctccaatgcattttccagtcagcacgtattgtattttttataagtagaaataaaacagtatcaaattcattttttttttttttttgtgttttggagtacattagttttgcgtatatttctttgtaaatatatgatctaccttattattttttgaactatgggattacctctattatatgctaatcatatttatcctgtttcattcatttgatatttttattttcaccgtcttctgctcttatatttttacgtctgtaataagttttgcgtatatttctttgtaaatatatgatctaccttattattttttgaactatgagattacctttattatatgctaatcatatttatcctgtttcattcatttgatatttttattttcaccgtcttctgctcttatatttttacttctataataagttaattatacgtctattatactttgatttttatccatttgatattttatatttatcattcggacttttgtaagtctaattttttccacaaacggtaattatttacaaaacatttttggtcaaaaatcgatgaaaaaaaatggtttctatttttcctcgcctattaggccaaccttcgtagaaaaagtatgaacggttactttataatgctaaagttattattaccctataatacctactcagctactcttacaagaaaatcaacgcatacataacttctcataaattatccacctactggaatatcaaatgtacaatatacagtaattagggaataagtcaataatgatttgtttttaatttttgtaaaaagtcaataatcaaatttaaggacgacacgaaaacaataatcaccattaaactatataacatcaatttaatacattggttagataaaaagaaatcgcgttattatattttatggttttaatttaaaaataatatttggaaaacagaggctctgctctctctctccctatctctgtaggtaccctcgttgctctctataagtactcccacaaccacgaactccaaaacatcatctcataaaccaaaaaccacattatccgagatttgagtatatttcactacaaccttcttgtcattttctctgtaaccatgg